(19) United States
(12) Patent Application Publication
(10) Pub. No.: US 2009/0211493 A1
(43) Pub. Date: Aug. 27, 2009

(54) Different Types Of Mineral Matter Containing Carbonate With Reduced Fossil Fuel Carbon Dioxide Emission On Breakdown, Together With Their Synthesis Process And Their Uses

(75) Inventors: Matthias Buri, Rothrist (CH); Thoralf Gliese, Buchrain (CH)

Correspondence Address: AMSTER, ROTHSTEIN & EBENSTEIN LLP, 90 PARK AVENUE, NEW YORK, NY, 10016, US

(73) Assignee: OMYA DEVELOPMENT AG, Oftringen (CH)

(21) Appl. No.: 11/920,525

(22) PCT Filed: May 11, 2006
(86) PCT No.: PCT/IB2006/001321

§ 371 Date: Feb. 11, 2009

(30) Foreign Application Priority Data
May 20, 2005 (FR) 05/05053
Nov. 25, 2005 (FR) 05/11921

(51) Int. Cl. C09D 1/00; C01B 31/24; C01D 15/08; C01F 5/24; C12P 3/00; C01F 11/18; C01D 1/00

(52) U.S. Cl. 106/417; 423/579; 423/641; 423/635; 106/486; 435/168; 106/482

(57) ABSTRACT

The invention concerns a new synthetic mineral matter containing carbonate, the decomposition of which reduces the rate of fossil fuel carbon dioxide emission.

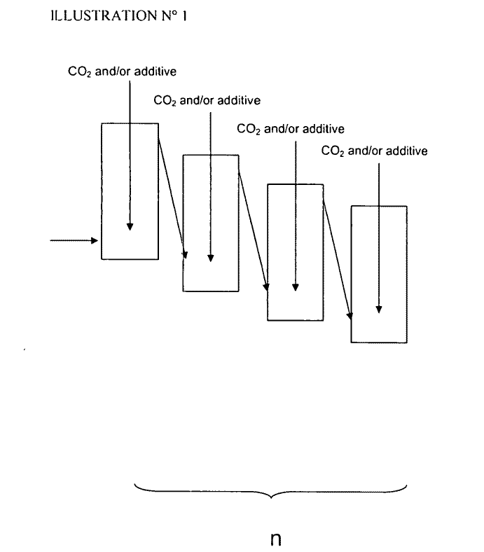

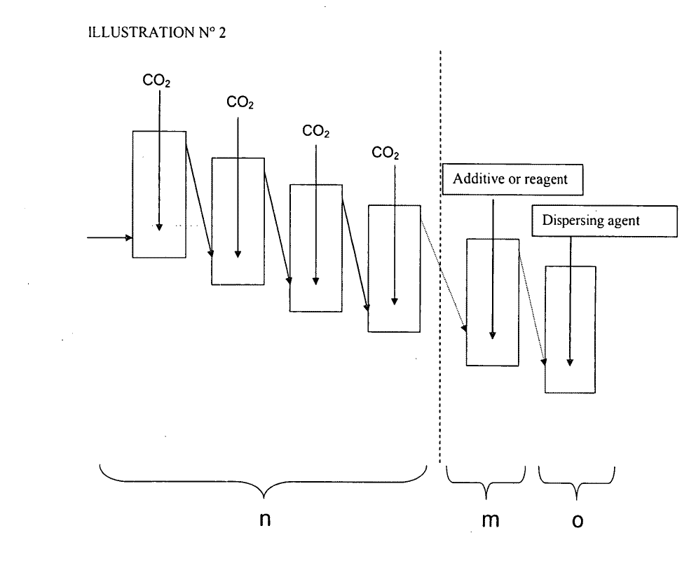

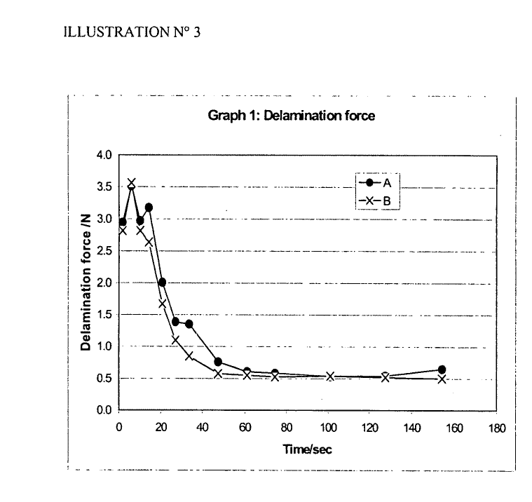

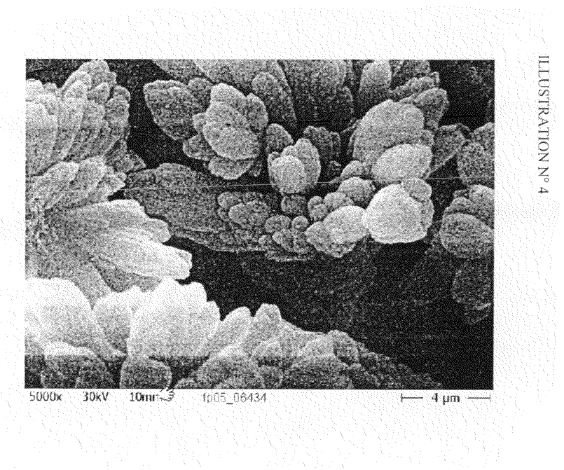

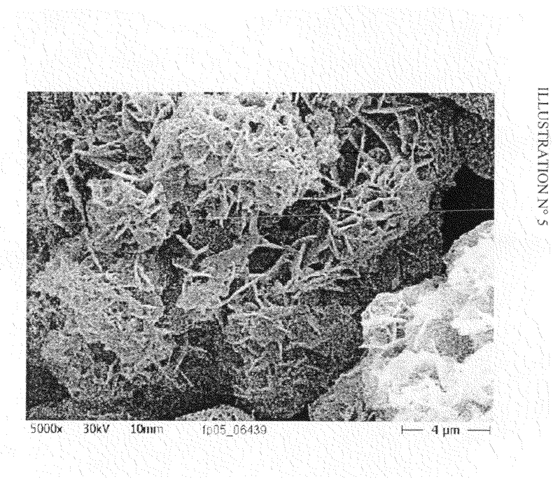

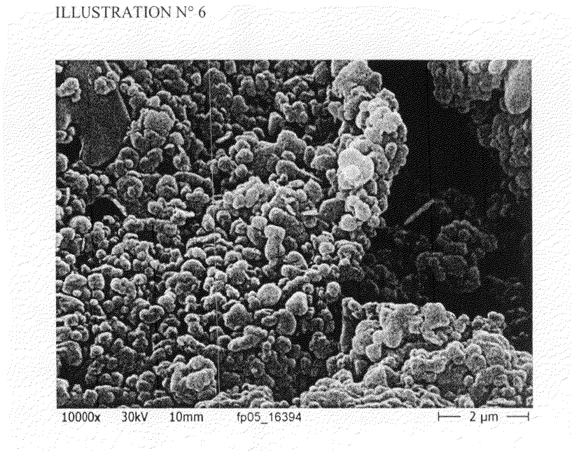

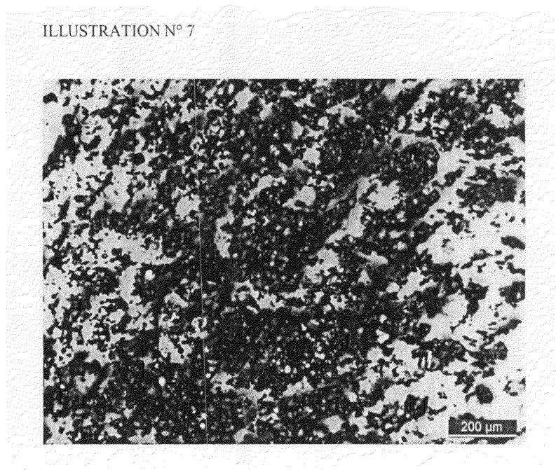

It also concerns its manufacture in batches, or in a batch-continuous manner, or in a continuous manner, together with its uses in the pharmaceutical field, the field of human or animal foodstuffs, or again the papermaking field with, notably, manufacture of paper, filler or coating, or again every other paper surface treatment, together with the fields of water-based or non-water-based paints, together with the field of plastics, such as that of breathable polyethylene films, or again the field of printing inks.

The present invention concerns the field of mineral matter (mineral fillers and/or pigments) containing carbonate, together with their sectors of application.

In these sectors of application, which are the pharmaceutical field with, notably, products such as medicines, the field of human or animal foodstuffs, or again the papermaking field with, notably, manufacture of paper, filler or coating, or again every other surface treatment of paper, together with the fields of water-based or non-water-based paints, together with the field of plastics and, notably, fillers for breathable polyethylene film, or again the field of printing inks, it is common to use, before, during or after production of these various products, natural or synthetic mineral matter (fillers and/or pigments) containing carbonate.

With the aim of protecting the environment and combating the greenhouse effect, and in the context of the Agreement on planetary climate change, a protocol relative to carbon dioxide atmospheric emissions was signed in Kyoto on 11 Dec. 1997 in order to reduce the quantity of these atmospheric carbon dioxide emissions.

In the aim of reducing these carbon dioxide emissions, approximately 80% of which result from the burning of coal, oil or natural gas, and 43% of which result from global emissions of the industrial sector, the Applicant has found, after much research, firstly new types of mineral matter (fillers and/or pigments) which, on breakdown, emit only small quantities of fossil fuel carbon dioxide, and secondly the process of their synthesis.

At this stage, it should be noted that by the term fossil fuel carbon dioxide, the Applicant means carbon dioxide originating in the main from the burning of fossil energies, such as coal, oil or natural gas, or the calcination of minerals.

Thus, the invention concerns a new synthetic mineral matter containing carbonate, the decomposition of which reduces the rate of fossil fuel carbon dioxide emission.

It also concerns the manufacture of this new mineral matter in batches, in cascade, also called in continuous fashion, or in a continuous-batch mixture, together with its uses in the various fields which are the pharmaceutical field, the field of human or animal foodstuffs, or again the papermaking field with, notably, manufacture of paper, filler or coating, or again every other paper surface treatment, together with the fields of water-based or non-water-based paints, together with the field of plastics, such as, notably, that of breathable polyethylene films, or again the field of printing inks.

The problem thus posed to the skilled man in the art, namely to comply with the Kyoto protocol in terms of atmospheric carbon dioxide emissions, has found no satisfactory solution in the prior art known at the present time by the Applicant.

Indeed, at the present time, the Applicant is aware of more than 100 patents relative to the synthesis of synthetic calcium carbonate, also called precipitated calcium carbonate, commonly known as “PCC”. These patents essentially describe either processes to synthesise synthetic calcium carbonate, based on the calcination of natural calcium carbonate, and the reuse of the released carbon dioxide, or processes based on the reaction of lime and carbon dioxide, the source of which originates from coal, oil or natural gas. In addition, a large majority indicates that the source of the carbon dioxide has no influence on the end product obtained.

Thus, the Applicant can, among these many patents, mention the American patents U.S. Pat. No. 6,251,356, U.S. Pat. No. 6,666,953, U.S. Pat. No. 6,579,410, U.S. Pat. No. 6,540,870, U.S. Pat. No. 6,540,878, U.S. Pat. No. 6,475,459, U.S. Pat. No. 6,440,209, U.S. Pat. No. 6,221,146, U.S. Pat. No. 6,416,727 or again documents WO 01/17905, EP 0 799 797, EP 1 222 146 or JP 08/252,595, which disclose the synthesis of PCC through the use of carbon dioxide resulting from the combustion of fossil energy, making no mention of carbon 14 (¹⁴C).

Moreover, a study undertaken on the reduction of carbon dioxide emissions (Possibilities of reducing CO₂ Emissions in the Finnish Forest Industry, S. Siitonen & P. Ahtila, Otaniemi 2002 published by Finnish Forest, Industries Federation, Helsinki 9/2002) leads the skilled man in the art to envisage using carbon dioxide originating from lime kilns to manufacture PCC and reduce the quantity of emitted carbon dioxide.

Thus, the skilled man in the art who must find a solution to reduce the emission of carbon dioxide originating from the burning of fossil energy had, currently, no satisfactory solution.

Faced with this problem, the Applicant has found, in an unexpected manner, that synthetic mineral matter containing carbonate having a high rate of carbon 14 (¹⁴C) enables the Kyoto protocol to be satisfied, thanks to the reduction of fossil fuel carbon dioxide emission.

Thus, the first aim of the invention is a new synthetic mineral matter containing carbonate having a high rate of carbon 14 (¹⁴C).

Another aim of the invention is a process to manufacture synthetic mineral matter containing carbonate having a high rate of carbon 14 (¹⁴C).

An additional aim of the invention is the use of synthetic mineral matter according to the invention in the abovementioned fields.

Throughout the present application, it should be noted that by the term mineral matter the Applicant means a mineral pigment and/or mineral filler.

Thus, synthetic mineral matter containing carbonate having a high rate of ¹⁴C according to the invention is characterised in that it has a rate of nuclear carbon transformation from ¹⁴C into ¹²C of at least 450 transformations per hour and per gram, and preferentially between 700 and 890 transformations per hour and per gram, and very preferentially between 850 and 890 transformations per hour and per gram.

Throughout the present application and in the claims, the rate of nuclear carbon transformation from ¹⁴C into ¹²C of the synthetic mineral matter is made according to a method of determining the rate of nuclear transformation, the originality of which lies in the sample preparation stage.

Indeed, the traditional methods for analysis of the rate of nuclear carbon transformation from ¹⁴C into ¹²C known hitherto are based on a stage of preparation consisting of a thermal decomposition at a high temperature (approximately 1000° C.) by combustion or calcination of the sample for analysis, followed by collection of the released carbon dioxide which is trapped at low temperature before its reduction, by catalytic hydrogenation, into elemental carbon atoms, the composition of which in ¹³C/¹²C and ¹⁵N/¹⁴N isotopes, and also ¹⁴C isotopes, is measured by a mass spectrophotometer.

However, it appears that these methods based on thermal decomposition of the sample for analysis do not enable any differentiation of the sources of the carbon dioxide, i.e. do not allow any differentiation of the carbon originating from the organic phases and the inorganic or mineral phases.

Thus, the Applicant has developed a method to determine the rate of nuclear transformation characterised by a stage of preparation of the sample using a donor of H₃O⁺, such as, notably, hydrochloric acid, or other donors of H₃O⁺ stronger than carbonic acid, such as notably phosphoric acid, and which enable only the carbon originating from the inorganic or mineral phase of the sample for analysis to be dosed.

This process to determine the rate of nuclear transformation from ¹⁴C into ¹²C of the synthetic mineral matter containing carbonate according to the invention is characterised in that it comprises:

a) a stage of preparation of the sample consisting of an attack of the sample by a donor of H₃O⁺, preferentially hydrochloric acid or any donor of H₃O⁺ stronger than carbonic acid such as phosphoric acid,
b) a stage of collection of the released carbon dioxide in a trap at the temperature of liquid nitrogen.
c) a stage of reduction of the carbon dioxide, by hydrogenation on an iron catalyst, into elementary carbon atoms (¹³C/¹²C/¹⁴C)
d) followed by analysis by radiological measurement, in particular by mass spectroscopy, of the composition in ¹³C/¹²C and ¹⁵N/¹⁴N and also ¹⁴C isotopes compared to an international reference standard enabling the rate of nuclear transformation per hour and per gram to be established.

A preferential variant of the process to determine the rate of nuclear transformation, characterised in that, between the stage of the acid attack and that of the collection of the released carbon dioxide, an additional trap is added having a temperature of 20° C. to 30° C. above the trap of stage b), in order to prevent any contamination by other volatile compounds originating from the sample.

The Applicant stresses that the rates of nuclear transformation of 450 transformations per hour and per gram, and preferentially between 700 and 890 transformations per hour and per gram, and very preferentially between 850 and 890 transformations per hour and per gram, which are the essential characteristic of the product forming the object of the invention, can however be determined by any other appropriate method.

The sample for analysis according to the invention consists of the mineral matter containing the carbonate according to the invention, and may be a sheet of paper containing the mineral matter, a calcium carbonate treated by organic compounds, a polyvinyl chloride (PVC) composition, a calcium carbonate having organic impurities, or any other sample.

In a particular manner, the mineral matter according to the invention is characterised in that the carbonate is chosen from among the carbonates with monovalent and/or bivalent and/or trivalent cations, or their mixtures.

In a more particular manner, this mineral matter according to the invention is characterised in that the said monovalent and/or bivalent and/or trivalent cations are chosen from among the cations of the first or second main group of the Mendeleev periodic table.

In a most particular manner, the said cations are chosen from among lithium, sodium, potassium, magnesium, calcium, strontium, or their mixtures.

The synthetic mineral matter containing a carbonate according to the invention is characterised preferentially in that it is a calcium carbonate having a crystalline structure of the calcite or aragonite or vaterite type, or in that it is a mixture of a calcium carbonate of structure of the calcareous type with a calcium carbonate of structure of the aragonite type and/or a calcium carbonate of structure of the vaterite type, and more preferentially in that it is a mixture of the structure of the calcite type and of the structure of the aragonite type.

In a more particular manner the said calcium carbonate according to the invention with the abovementioned crystalline structure is characterised in that it has a degree of whiteness of over 80%, and preferentially over 90%, and very preferentially over 93% TAPPI, determined according to norm TAPPI T452 ISO 2470.

In another variant of the invention, the synthetic mineral matter containing carbonate according to the invention is characterised in that it is a mixture and/or a costructure of the abovementioned carbonates with other types of mineral matter chosen from among the natural and/or synthetic silicas, the silicates such as notably clay, talc, mica, or again chosen from among the aluminium hydroxides, the sulphates, the satin whites, the phosphates such as the brushites, octacalcium phosphates or the hydroxyapatites, or their mixtures.

The process of manufacture of the mineral matter according to the invention is characterised in that it uses carbon dioxide resulting from the aerobic or anaerobic fermentation, preferentially anaerobic, and more particularly resulting from the fermentation of sugars or from the combustion of alcohol deriving from the fermentation of organic compounds.

In the remainder of the present application, the Applicant stipulates that the carbon dioxide resulting from the aerobic or anaerobic fermentation or fermentation of sugars or from the combustion of alcohol resulting from the fermentation of organic compounds will be called fresh carbon dioxide, unlike fossil fuel carbon dioxide resulting from the combustion of fossil energies such as coal, oil or natural gas, or again carbon dioxide resulting from the calcination of natural calcium carbonate, which will be called old carbon dioxide.

In a particular manner, fresh carbon dioxide results from the fermentation of sugars or from the combustion of alcohol, particularly ethanol, methanol or alkanes such as methane, ethane or any other alkane, resulting from the fermentation of organic compounds such as fruit, fruit alcohols or again waste from public discharges, or results from fermentation or thermal decomposition, or from degradation by oxidation of waste from discharges under supercritical pressure.

Another particular manner consists in that the fresh carbon dioxide used in the process according to the invention is a mixture of fresh carbon dioxide resulting from the fermentation of sugars with fresh carbon dioxide resulting from the combustion of organic compounds.

Another variant of the process according to the invention is characterised in that it uses a mixture carbon dioxide resulting from the aerobic or anaerobic fermentation, preferentially anaerobic, and more particularly resulting from the fermentation of sugars or from the combustion of alcohol deriving from the fermentation of organic compounds, with old carbon dioxide. According to this variant, the process according to the invention is characterised in that the mixture uses less than 50% by weight of old carbon dioxide.

In a more particular manner, the process of manufacture of synthetic mineral matter containing carbonate according to the invention is characterised in that the carbon dioxide is used at a temperature of between 5° C. and 100° C., and preferentially between 20° C. and 30° C.

This process according to the invention is characterised in that it is a batch process, in cascades, otherwise called continuous, or in a continuous-batch mixture.

By batch process, the Applicant means a manufacturing process in which the reaction takes place in a single tank into which all the reagents are introduced.

By a process in cascades, otherwise called continuous, the Applicant means a manufacturing process in which the fresh carbon dioxide used is introduced in a cascade of n reactors installed in series and/or in parallel, the arrangement in series of which is illustrated by illustration n^{o} 1.

In this illustration n^{o} 1, the Applicant designates by “n” the number of reactors in cascades, into which are introduced continuously the carbon dioxide and/or any other additive, this number n ranging from 1 to 50, and preferentially from 1 to 10, and very preferentially from 1 to 5.

By a process in a continuous-batch mixture, the Applicant means a continuous synthesis process followed by one or more batch stages, where this or these latter stages may be the addition of carbon dioxide to the storage or the addition of various additives, or again a stage of physical processing (such as grinding, centrifugation, thermal concentration, mechanical concentration) or a stage of chemical processing such as treatment with sodium silicate followed by the addition of an acid such as citric acid or phosphoric acid, and possibly at least one stage of introduction of a dispersing agent.

In illustration n^{o} 2 illustrating this continuous-batch mixture process, the Applicant designates by “m” the number of the abovementioned physical or chemical treatments, where this or these treatments may be in particular a treatment by mechanical concentration by use of a centrifuge.

This number “m” varies from 1 to 5, and preferentially from 1 to 2. Similarly, in this illustration n^{o} 2, the Applicant indicates by “o” the number of stages corresponding to a possible addition of dispersing agent, where this dispersing agent is any type of dispersing agent known to the skilled man in the art, the choice of which is chosen in an obvious manner by the skilled man in the art in accordance with the envisaged application.

This number “o” varies from 0 to 3, and preferentially from 0 to 1.

Finally, the process according to the invention is characterised in that it consists possibly of at least one stage of dispersion and/or at least one stage of grinding in a dry medium or a wet medium, in the presence of possibly at least one dispersing agent and/or at least one grinding aid agent. The skilled man in the art will know how to adapt the choice of any dispersing agents and/or grinding aid agents he may use in accordance with the final envisaged application.

Finally, the mineral matter according to the invention is used in the pharmaceutical field with products such as medicines, the field of human or animal foodstuffs, or again the papermaking field such as the manufacture of paper, filler and/or coating on a paper or plastic support, or again any other paper and/or plastic surface treatment, where the plastic is preferentially chosen from among the polyolefins of the polyethylene or polypropylene type and their derivatives, together with the fields of water-based or non-water-based paints, and the field of plastics, or again the field of printing inks.

The use of mineral matter in paper coating is made preferentially during coating operations by blade, film transfer, water wash, or again by “size-press”.

The use of mineral matter as a paper mass filler is made preferentially by the addition of mineral matter according to the invention in different locations before and/or during formation of the sheet.

The mineral matter according to the invention used in the field of printing inks is used in inks for inkjet printing, for offset printing and/or rotogravure printing.

The scope and interest of the invention will be better appreciated through the following examples, which are by no means limitative.

### EXAMPLE 1

This example illustrates different processes of the prior art using an old carbon dioxide.

### Test N^{o} 1

This test, which illustrates the prior art, concerns a process of manufacture of calcium carbonate precipitated by reaction of lime with old carbon dioxide resulting from the combustion of fossil energy such as butane.

To accomplish this, after having produced calcium oxide CaO by calcination of a ground natural calcium carbonate (Omyapure™ from the company Omya SAS) in a muffle kiln for 6 hours at 900° C., the calcium oxide CaO is put in suspension in a container containing water without carbon dioxide to form a suspension of calcium hydroxide Ca(OH)².

Once this suspension of lime has been formed with a weight concentration of dry matter equal to 10%, old carbon dioxide, resulting from the combustion of butane using a camping gas burner, is introduced into it at ambient temperature (22° C.±2° C.) until the pH falls from a strongly alkaline value of around 13 to a value of between 8.0 and 8.5.

The product obtained, which is a precipitated calcium carbonate of the prior art, is then dried at 140° C.

### Test N^{o} 2

This test, which illustrates the prior art, concerns a process of manufacture of calcium carbonate precipitated by reaction of lime with old carbon dioxide resulting from the calcination of natural calcium carbonate.

To accomplish this, with the same equipment and the same operating method as in the previous test, old carbon dioxide, resulting from the calcination in a muffle kiln at 900° C. of a ground natural calcium carbonate (Omyapure™ from the company Omya SAS), is made to react with a suspension of lime at 10% by weight of dry matter.

The product obtained, which is a precipitated calcium carbonate of the prior art, is then dried at 140° C.

### Test N^{o} 3

This test, which illustrates the prior art, concerns a process of manufacture of strontium carbonate precipitated by reaction of strontium hydroxide with old carbon dioxide resulting from the calcination of natural calcium carbonate.

To accomplish this, strontium hydroxide Sr(OH)₂×8 (H₂O), (Batch 9329A from Riedel-de Haën), is put in suspension in a container containing water without carbon dioxide to form a suspension of strontium hydroxide at a dry matter concentration equal to 10% by weight.

Once this suspension has been formed, old carbon dioxide, resulting from the calcination in a muffle kiln at 900° C. of a ground natural calcium carbonate (Omyapure™ from the company Omya SAS), is introduced into it at ambient temperature (22° C.±2° C.), until the pH falls to a value of between 8.0 and 8.5.

The product obtained, which is a strontium carbonate of the prior art, is then dried at 140° C.

### EXAMPLE N^{o} 2

This example illustrates the process according to the invention using fresh carbon dioxide.

### Test N^{o} 4

This test, which illustrates the invention, concerns a process of manufacture of calcium carbonate precipitated by reaction of lime with fresh carbon dioxide resulting from the combustion of ethanol originating from the fermentation of an organic compound such as kirsch.

To accomplish this, after having produced calcium oxide CaO by calcination of a ground natural calcium carbonate (Omyapure™ from the company Omya SAS) in a muffle kiln for 6 hours at 900° C., the calcium oxide CaO is put in suspension in a container containing water without carbon dioxide to form a suspension of calcium hydroxide Ca(OH)₂.

At the same time, a kirsch alcoholic drink purchased from a supermarket was distilled from 37% by volume to 65% by volume and was then treated with sodium sulphate to absorb the remainder of the water and obtain a concentration of 97% ethanol.

Once this suspension of lime has been formed and this alcoholic drink obtained, the fresh carbon dioxide, resulting from the combustion of the abovementioned ethanol, using a methanol burner, is introduced into the lime suspension at ambient temperature (22° C.±2° C.), until the pH falls to a value of between 8.0 and 8.5.

The product obtained, which is a precipitated calcium carbonate according to the invention, is then dried at 140° C.

### Test N^{o} 5

This test, which illustrates the invention, concerns a process of manufacture of strontium carbonate by reaction of strontium hydroxide with fresh carbon dioxide resulting from the combustion of ethanol originating from the fermentation of an organic compound such as kirsch.

To accomplish this, strontium hydroxide Sr(OH)₂×8 (H₂O), (batch 9329A from Riedel-de Haën), is put in suspension in a container containing water without carbon dioxide to form a suspension of strontium hydroxide at a dry matter concentration equal to 10% by weight.

At the same time, a kirsch alcoholic drink purchased from a supermarket was distilled from 37% by volume to 65% by volume and was then treated with sodium sulphate to absorb the remainder of the water and obtain a concentration of 97% ethanol.

Once this suspension strontium hydroxide Sr(OH)₂×8 (H₂O) is formed and this alcoholic drink obtained, the fresh carbon dioxide, resulting from the combustion of the abovementioned ethanol, using a methanol burner, is introduced to the strontium hydroxide suspension at ambient temperature (22° C.±2° C.), until the pH falls to a value of between 8.0 and 8.5.

The product obtained, which is a strontium carbonate according to the invention, is then dried at 140° C.

### Test N^{o} 6

This test, which illustrates the invention, concerns a process of manufacture by batch of calcium carbonate precipitated by reaction of lime with fresh carbon dioxide resulting from the fermentation of a sugar.

To accomplish this, after having produced calcium oxide CaO by calcination of a ground natural calcium carbonate (Omyapure™ from the company Omya SAS) in a muffle kiln for 6 hours at 900° C., 30 g of the calcium oxide CaO is put in suspension in a beaker containing 200 g of water without carbon dioxide to form a suspension of calcium hydroxide Ca(OH)₂.

In addition, 1 kg of refined household sugar (C₁₂H₂₃O₁₁), which is dissolved in 4 litres of distilled water, to which is added 7 dry g of baker's yeast, to allow the release of carbon dioxide resulting from the fermentation of the sugar, is introduced into a 5-litre recipient.

This fresh carbon dioxide which is created, and which is continually formed for 21 days by fermentation of the sugar, is then introduced into the lime suspension during these 21 days at ambient temperature (22° C.±2° C.) until the pH falls as far as a value of approximately 7±0.3.

This introduction of fresh carbon dioxide into the lime suspension is accomplished by means of a distilled water washing recipient which collects any evaporated ethanol.

The product obtained, which is a precipitated calcium carbonate according to the invention, is then dried at 140° C.

### Test N^{o} 7

This test, which illustrates the invention, concerns a process of continuous manufacture of calcium carbonate precipitated by reaction of lime with fresh carbon dioxide resulting from the fermentation of a sugar.

To accomplish this, an aqueous suspension of lime and fresh carbon dioxide resulting from the fermentation of a mixture of 500 g of saccharose and 42 g of baker's yeast (saccharomyces cerevisiae) is introduced into 4 litres of water in a cascade of 4 reactors filled with distilled water and installed in series, as illustrated by illustration n^{o} 1.

Thus, firstly an aqueous suspension of lime is prepared by putting in suspension 1000 g of calcium hydroxide in a recipient containing 50 litres of water without carbon dioxide, stirred by a mechanical stirrer.

4 closed 5-litre flasks are then arranged, in each of which a mixture of 500 grams of refined household sugar (C₁₂H₂₃O₁) and 42 grams of baker's yeast are dissolved in 4 litres of distilled water to enable the release of carbon dioxide gas resulting from the fermentation of sugar for 5 days.

When these operations have been completed, the following operations are commenced simultaneously: the previously formed carbon dioxide gas is introduced into each of the flasks, and the lime suspension, contained in the recipient stirred by a mechanical stirrer in continuous fashion in the 4 reactors fitted with a lid and linked to one another by means of a pipe, is pumped.

The rate of introduction of the lime suspension is an introduction of 7.35 g by dry weight of lime per hour until 45 of the 50 litres of lime suspension have been introduced into the 4 carbonation reactors.

Thus, the fresh carbon dioxide, which is created and continuously formed for 5 days by fermentation of sugar, is introduced into the 4 reactors stirred at 400 rpm and at a temperature equal to 25° C.±3° C. for 5 days.

The pH in the fourth reactor has a value of between 6.7 and 7.3.

This introduction of fresh carbon dioxide into the lime suspension is accomplished by means of a distilled water washing recipient which collects any evaporated ethanol.

The product obtained, which is recovered in a final tank, is a precipitated calcium carbonate according to the invention, is then dried at 140° C.

This precipitated calcium carbonate obtained is a pure calcite as it is shown by the infrared spectrum and the following granulometric distribution, expressed as a percentage of weight of particles, and measured with a granulometer of the Sedigraph™ 5100 type:
77% have a diameter <2 μm,
44% have a diameter <1 μm,
6% have a diameter <0.2 μm,

### Test N^{o} 8

This test, which illustrates the invention, concerns a process for continuous manufacture of a precipitated calcium carbonate obtained by reaction of lime with fresh carbon dioxide resulting from the fermentation of a sugar, and subsequently processed in a fifth reactor by a sodium silicate.

To accomplish this, a cascade of 5 reactors arranged in series is installed, as illustrated in illustration n^{o} 2.

In the cascade of the first 4 reactors, the product obtained with the same operating method as the previous test is a precipitated calcium carbonate according to the invention, which is then treated in the fifth reactor by sodium silicate (Inosil 4237 from Van Berle), diluted in water at 1% by weight, in a quantity equivalent to 4% by dry weight relative to the dry weight of calcium carbonate formed. The dosage was set at 0.22 g of sodium silicate per hour, corresponding to 22 ml per hour of a solution of 1% by weight.

The pH is then 10.8±0.1 in the fifth reactor at the end of the test.

### Test N^{o} 9

This test, which illustrates the invention, concerns a process for continuous manufacture of a precipitated calcium carbonate obtained by reaction of lime with fresh carbon dioxide resulting from the fermentation of a sugar, and subsequently processed in the penultimate reactor by a sodium silicate, and in the final reactor by the addition of citric acid.

To accomplish this, a cascade of 6 reactors arranged in series is installed, as illustrated in illustration n^{o} 2.

In the cascade of the first 4 reactors, the product obtained with the same operating method as the previous test is a precipitated calcium carbonate according to the invention, which is then treated in the fifth reactor by sodium silicate (Inosil 4237 from Van Berle), in a quantity equivalent to 4% by dry weight relative to the dry weight of calcium carbonate formed. The pH is then 10.8±0.1.

In the sixth reactor, which allows the pH to be inspected and adjusted, the necessary quantity of citric acid to obtain a pH of value equal to 8.5±0.3 is then added in continuous fashion.

It should be noted that in this example the addition into this sixth reactor could, in an equivalent manner, be undertaken in batch fashion.

### Test N^{o} 10

This test, which illustrates the invention, concerns a process of manufacture by batch of a sodium carbonate by reaction of soda with fresh carbon dioxide resulting from the fermentation of a sugar, followed by the manufacture of a precipitated calcium carbonate, by reaction of the sodium carbonate formed according to the invention with a calcium chloride.

To accomplish this, firstly 30 g of sodium hydroxide for analysis sold by Riedel-de Haën is dissolved in 120 g of water without carbon dioxide and 500 g of refined household sugar (C₁₂H₂₃O₁₁) is then introduced into a 2.5 litre recipient, which is dissolved in 2 litres of distilled water, to which is added 21 g of fresh baker's yeast, corresponding to 7 dry g of baker's yeast in order to allow the release of carbon dioxide resulting from the fermentation of sugar.

This fresh carbon dioxide which is created, and which is continually formed for 21 days by fermentation of the sugar, is then introduced into the soda solution during these 21 days at ambient temperature (22° C.±2° C.) until the pH falls to a value of between 8.0 and 8.5.

This introduction of fresh carbon dioxide into the soda solution is accomplished by means of a distilled water washing recipient which collects any evaporated ethanol.

Part of the sodium carbonate solution obtained is then filtered using a 0.45 μm filter to separate the insoluble components and obtain a filtrate which is then dried at 140° C. to obtain the sodium carbonate according to the invention.

In a second stage, the sodium carbonate according to the invention is then mixed at ambient temperature (22° C.±2° C.) with a stoichiometric quantity of calcium chloride to obtain a precipitated calcium carbonate, the insolubles of which are filtered as before, and the filtrate of which is dried at 140° C.

### Test N^{o} 11

This test, which illustrates the invention, concerns a dry mixture of precipitated calcium carbonate obtained by the mixing of a dry calcium carbonate according to the invention (test n^{o} 4) with a dry calcium carbonate of the prior art (test n^{o} 2) in a weight ratio of 55/45.

### Test N^{o} 12

This test, which illustrates the invention, concerns a dry mixture of precipitated calcium carbonate obtained by drying of a mixture of a suspension of calcium carbonate according to the invention (test n^{o} 4) with a calcium carbonate suspension of the prior art (test n^{o} 2) in a weight ratio of 51/49.

### EXAMPLE 3

This example illustrates the process according to the invention for determining the rate of nuclear transformation from ¹⁴C into ¹²C of a medicinal formulation essentially consisting of calcium carbonate.

### Test N^{o} 13

This test, which illustrates the prior art, uses a dry medicinal formulation consisting of a powder of ground natural calcium carbonate sold by the company Omya SAS under the name Omyapure™.

To accomplish this, 30 mg of the sample for testing is placed in a 7 ml phial equipped with two consecutive 14.7 ml reactors at a pressure of 250 mbar, thus forming 2 consecutive traps, the latter of which is a liquid nitrogen trap and the first of which is cooled to a temperature 20° C. to 30° C. above the second, to prevent all contamination by other volatile compounds deriving from the sample.

Approximately 0.4 ml of hydrochloric acid is then poured into this phial, which will then react with the medicinal formulation and release carbon dioxide which will be trapped in the successive traps.

The carbon dioxide trapped in this manner is then reduced by hydrogenation on a cobalt powder catalyst into elementary carbon atoms (¹³C/¹²C/¹⁴C).

The ¹³C/¹²C and ¹⁵N/¹⁴N isotope composition of the graphite thus obtained is then determined, together with that of ¹⁴C compared to an international reference standard by the technique of accelerator mass spectrometry, known as the “AMS” technique (“Accelerator Mass Spectrometry”) using a spectrophotometer commonly used by the skilled man in the art.

The rate of nuclear transformation per hour and per gram of the synthetic mineral matter for analysis is then determined by the ratio between the ¹⁴C value measured and the value of the reference standard recognised internationally and referenced in the methods for dating by ¹⁴C.

### Test N^{o} 14

This test, which illustrates the invention, uses, with the same operating method and the same equipment as the previous test, a dry medicinal formulation consisting of a calcium carbonate according to test n^{o} 4.

### EXAMPLE 4

This example illustrates the use of types of mineral matter according to the invention in the papermaking field, together with the process according to the invention for determining the rate of nuclear transformation from ¹⁴C into ¹²C of a formulation in the papermaking field, and more particularly in the field of “offset” ink.

### Test N^{o} 15

This test illustrates the prior art and uses, in an “offset” ink application, a precipitated calcium carbonate of test n^{o} 1.

To accomplish this, using a vibrating disk grinder of type HSM 100-H sold by the company Herzog, fitted with a tungsten carbide grinding chamber with internal diameter of 95 mm, filled with a 60 mm diameter grinding body, 20 g of the precipitated calcium carbonate of test n^{o} 1 is ground to a fineness, of which the granulometric characteristics determined by measurement using a Sedigraph™ 5100 granulometer from Micromeritics™, are:

average diameter of approximately 1.8 μm,

91.9% by weight of particles have a diameter of less than 5 μm,

56.8% by weight of particles have a diameter of less than 2 μm,

25.9% by weight of particles have a diameter of less than 1 μm,

9.7% by weight of particles have a diameter of less than 0.5 μm,

4.0% by weight of particles have a diameter of less than 0.2 μm.

Then, using a mortar equipped with a “pistil”, this 20 g of ground precipitated calcium carbonate is added to and dispersed in 200 g of an “offset” ink sold by the company Schaffner GF AG (Switzerland) under the name Skinex Cyan 4X800.

The composition then produced is used to print an IKONOFIX™ paper, 150 g/m², from M-Real™ Zanders GmbH, Bergisch-Glattbach (Germany) using a laboratory “offset” printing machine sold by the company SeGan (Great Britain) under the name Ink/Surface Interaction Tester.

This test, called an ISIT (Ink Surface Interaction Test) printability test, which represents the ink delamination force as a function of time, is a graph with three phases: a rising phase with a distinct upward slope, a maximum value, followed by a descending phase with a distinct downward slope, and is based on a printing installation fitted with a device to create and measure the necessary force to separate a delamination disk, from a printing ink film. This installation, consisting firstly of this device for creating and measuring force, and secondly of an inking disk rotating above the sheet of paper for testing is sold under the name “Ink Surface Interaction Tester” by the company SeGan Ltd.

To accomplish this, firstly the different sheets of paper for testing are prepared by applying the different coating colours for testing on to these sheets of paper using the Erichsen™ laboratory coating machine Model 624 from the company Erichsen™ GmbH+Co. KG (Germany), fitted with exchangeable rolling blades.

The paper thus coated has a determined value in g/m². It is fixed on to a roller fitted with a twin-face adhesive strip. An offset ink is applied by bringing the 25 mm wide inking disk into contact during a 180° rotation. The printing speed and pressure are adjustable and are around 0.5 m/s and 50 kg respectively. The ink volume is in the standard conditions 0.3 cm³, thus resulting in a thickness of approximately 1 g/m² of ink on the sheet of paper for testing.

The printing process is followed by a sequence of repeated measurements of the delamination force, at pre-selected time intervals depending on this time taken to separate this delamination disk (of the same dimension as the printing disk) from the ink film.

A nitrile rubber covering of offset printing quality is habitually used for the delamination disk, but any equivalent material may be used.

The contact force between the delamination disk and the ink is measured by a system generating an electromagnetic force. The amplitude and duration of the delamination force are adjusted to arrive at a uniform adhesion between the surface of the film and the delamination disk after 3 seconds. A small rotation of the sheet of paper during application of the electromagnetic force enables intimate contact and continuity of the ink film to be ensured. When the magnetic force is stopped, the delamination disk is retracted from the printed film by the force of a stretched spring; this is sufficient force to separate the disk from the ink film. A stress gauge, fixed between the delamination disk and the spring, generates a signal which is recorded as the delamination force.

The sequence is repeated automatically for 13 cycles.

In the first and thirteenth cycles the printing densities are measured using a Gretag D 186 densitometer.

When this measurement has been made, the rate of nuclear transformation from ¹⁴C into ¹²C of the paper sample thus printed is determined by putting 150 mg of the 20% filled sample of printed paper in a 7 ml phial equipped with two consecutive 14.7 ml reactors at a pressure of 250 mbar, thus forming 2 consecutive traps, the latter of which is a liquid nitrogen trap, and the first of which is cooled to a temperature 20 to 30° C. above the second in order to prevent all contamination by other volatile compounds deriving from the sample.

Approximately 0.4 ml of hydrochloric acid is then poured into this phial, which will then react with the sheet of printed paper and release carbon dioxide which will be trapped in the successive traps.

The carbon dioxide trapped in this manner is then reduced by hydrogenation on a cobalt powder catalyst into elementary carbon atoms (¹³C/¹²C/¹⁴C).

The ¹³C/¹²C and ¹⁵N/¹⁴N isotope composition of the graphite thus obtained is then determined, together with that of ¹⁴C compared to an international reference standard by the technique of accelerator mass spectrometry, known as the “AMS” technique (“Accelerator Mass Spectrometry”) using a spectrophotometer commonly used by the skilled man in the art.

The rate of nuclear transformation per hour and per gram of the synthetic mineral matter for analysis is then determined by the ratio between the ¹⁴C value measured and the value of the reference standard recognised internationally and referenced in the methods for dating by ¹⁴C.

### Test N^{o} 16

This test illustrates the invention and uses, in an “offset” ink application, precipitated calcium carbonate of test n^{o} 4.

To accomplish this, using a vibrating disk grinder of type HSM 100-H sold by the company Herzog, fitted with a tungsten carbide grinding chamber with internal diameter of 95 mm, filled with a 60 mm diameter grinding body, 20 g of the precipitated calcium carbonate of test n^{o} 4 is ground to a fineness, of which the granulometric characteristics determined by measurement using a Sedigraph™ 5100 granulometer from Micromeritics™, are:

an average diameter of approximately 1.7 μm,

93.2% by weight of particles have a diameter of less than 5 μm,

58.5% by weight of particles have a diameter of less than 2 μm,

26.8% by weight of particles have a diameter of less than 1 μm,

110.1% by weight of particles have a diameter of less than 0.5 μm,

4.2% by weight of particles have a diameter of less than 0.2 μm.

Then, using a mortar equipped with a “pistil”, this 20 g of ground precipitated calcium carbonate is added to and dispersed in 200 g of an “offset” ink sold by the company Schaffner GF AG (Switzerland) under the name Skinex Cyan 4×800.

The composition then produced is used to print an IKONOFIX™-type paper, 150 g/m², from M-Real™ Zanders GmbH, Bergisch-Glattbach (Germany) using a laboratory “offset” printing machine sold by the company SeGan™ (Great Britain) under the name Ink/Surface Interaction Tester, under the same conditions as those of the previous test.

The results obtained are shown in graph 1, which represents the ink delamination force as a function of time. This graph is represented in illustration n^{o} 3.

Graph 1 enables it to be recorded that the offset printing of the ink filled with calcium carbonate according to the invention of test n^{o} 16 (product B of graph 1) is identical to the offset printing of the ink filled with calcium carbonate according to the prior art of test n^{o} 15 (product A of graph 1).

### EXAMPLE 5

This example illustrates the use of types of mineral matter according to the invention in the field of polymer plastics, and in particular their use for the preparation of filled thermoplastic compositions such as, notably, filled polyvinyl chloride (PVC) compositions.

### Test N^{o} 17

This test illustrates the prior art and uses the precipitated calcium carbonate of test n^{o} 1. The filled PVC composition is produced by mixing non-filled PVC resin and calcium carbonate for dispersion in a Colling™-type grinder fitted with two rollers of diameter equal to 150 mm and length equal to 400 mm so as to obtain a pigment content of approximately 20% by weight.

In all the tests of the example, the formulation of the filled PVC composition is as follows:

| | |
|---|---|
| Corvic ™ S 704 | 100 parts |
| DIDP (diisodecyl phthalate) | 55 parts |
| Baropan E-292 | 4 parts |
| Carbonate for dispersion | 40 parts |

The precise calcium carbonate content equal to 19.7% by weight was determined by calcination for 2 hours at 650° C.

### Test N^{o} 18

This test illustrates the invention and uses the precipitated calcium carbonate of test n^{o} 4 by using the same operating method and the same equipment as the previous test.

The precise calcium carbonate content equal to 19.5% by weight was determined by calcination for 2 hours at 650° C.

Test N^{o} 19 This test illustrates the invention and uses a mixture of 50% by weight of the precipitated calcium carbonate of test n^{o} 4 and 50% by weight of precipitated calcium carbonate of test n^{o} 5, by using as a coating a polypropylene support.

5 g of precipitated calcium carbonate of test n^{o} 4 and 5 g of precipitated calcium carbonate of test n^{o} 5 were mixed and dispersed in 40 g of water with 0.1% by mass relative to the dry weight of fillers of a sodium polyacrylate (of molecular weight Mw equal to 3500 Daltons, and of polydispersity index equal to 2.7) as a dispersing agent. 2 g of a latex of type Acronal™ S 360 D, BASF™ (50% by mass of active product), were used as a binder.

Various quantities per m² of this coating colour were coated on a white, semi-transparent polypropylene film of type Synteape™, Fischer Papier™, St. Gallen-Rotkreuz (Switzerland). The whiteness and the opacity were determined.

The opacity is measured according to norm DIN 53146 and an Elrepho 2000 spectrophotometer of Datacolor™ AG (Switzerland) was used.

The whiteness called the Tappi R 457 whiteness is determined according to norm TAPPI T452 ISO 247.

The results are shown in table 2.

TABLE 2
coat weights and values of opacities and TAPPI
R 457 whitenesses measured respectively according
to norms DIN 53146 and TAPPI T452 ISO 247.

| | | | Gross TAPPI | Net TAPPI |
|---|---|---|---|---|
| Weight of | Gross | Net | R 457 | R 457 |
| coat (g/m²) | opacity | opacity | whiteness | whiteness |
| 0 | 87.79 | 0 | 89.44 | 0 |
| 1.69 | 87.97 | 0.18 | 91.05 | 1.61 |
| 4.16 | 88.96 | 1.17 | 91.16 | 1.72 |
| 8.4 | 90.16 | 1.37 | 91.37 | 1.93 |
| 15.25 | 90.87 | 3.08 | 91.53 | 2.09 |
| 19.8 | 91.97 | 4.18 | 91.81 | 2.37 |
| 23.22 | 92.42 | 4.63 | 91.97 | 2.53 |

The net values determined at a given coat weight correspond to the difference between the gross value measured for this coat weight and the gross value measured at a coat weight equal to 0 g/m².

These results show that more than 1.5 whiteness points are gained with a coat weight equal to 1.7 g/m².

More than 1.5 whiteness and opacity points are gained with a coat weight equal to 4 g/m².

With a coat weight of 20 g/m² more than 4 opacity points are gained.

### Test N^{o} 20

Using the calcium carbonate of test n^{o} 10, a suspension of the said carbonate in water is produced with a content of 10% by weight of dry matter, in the presence of 1% by weight of sodium polyacrylate relative to the dry weight of calcium carbonate as a dispersing agent. After 5 minutes of mechanical stirring the suspension is heated to 65° C. whilst stirring and 25% by mass of phosphoric acid relative to the dry weight of calcium carbonate is introduced. The phosphoric acid was dosed for 20 min. in the form of a 10% active solution. The temperature during the reaction was equal to 65° C.±5° C. After dosing, the reaction continued for 5 hours. The final pH measured at 23° C. was equal to 7.6.

The microscopic structures of the products obtained before and after treatment with phosphoric acid were photographed using an electronic microscope, and are shown respectively in illustrations n^{o} 4 and n^{o} 5.

The product finally obtained has a BET specific area equal to 52 m²/g.

This measurement of BET specific area is determined according to the BET method of norm ISO 9277, i.e. the measurement is made under cooling in liquid nitrogen and in a nitrogen current over the dried sample until a constant weight is obtained, and maintained at a constant temperature of 250° C. for one hour in a nitrogen atmosphere.

Finally, the rates of nuclear transformation from ¹⁴C into ¹²C were determined for each of the types of mineral matter previously exemplified.

To accomplish this, the same method and the same equipment were used as in example 3, except for one of the two comparative tests relative to the UPM™ Schongau paper, for which there is only a single trap, the liquid nitrogen trap.

The results obtained for the different products have been brought together in table n^{o} 2 below, which also groups together the results of the comparative tests made with pigments well known to the skilled man in the art.

These comparative tests use Socal™ P2 PCC sold by Solvay™, Type A PCC sold by Schaefer™, Syncarb™ F0474-GO PCC from Unikristall™ and a UPM™ Schongau paper.

TABLE
n° 2

| | | Rate of nuclear |
|---|---|---|
| | | transformation per |
| Prior art/ | | hour and per gram |
| Invention | Test n° | of mineral matter |
| Prior art | 1 | 33 ± 1 |
| Prior art | 2 | 25 ± 1 |
| Prior art | 3 | 28 ± 1 |
| Prior art | Comparison: Socal ™ PCC | 15 ± 1 |
| | Solvay ™ P2 | |
| Prior art | Comparison: Type A PCC | 8.5 ± 1 |
| | of Schaefer ™ | |
| Prior art | Comparison: Syncarb ™ | 27.5 ± 1 |
| | F0474-GO PCC of | |
| | Unikristall ™ | |
| Prior art | Comparison UPM ™ | 431 ± 4 * |
| | Schongau paper | 436 ± 4 ** |
| Invention | 4 | 866 ± 5 |
| Invention | 5 | 869 ± 5 |
| Invention | 6 | 879 ± 5 |
| Invention | 10: Na carbonate | 885 ± 5 |
| Invention | 10: Ca carbonate | 885 ± 5 |
| Invention | 11 | 476 ± 5 |
| Invention | 12 | 454 ± 5 |
| Prior art | 13 | 33 ± 1 |
| Invention | 14 | 866 ± 5 |

* with a method with a single liquid nitrogen trap
** with a method using both traps

### EXAMPLE 6

### Test N^{o} 21

This test, which illustrates the invention, concerns a process of continuous manufacture of calcium carbonate precipitated by reaction of lime with fresh carbon dioxide, where the said gas derives from the decomposition of an organic carbon source under a supercritical condition.

In addition, this test illustrates the use of one or more chemical and/or physical treatments, in the manufacture of precipitated calcium carbonate according to the invention.

To accomplish this, an aqueous suspension is prepared having a concentration equal to 15% by dry weight of lime, stirred by means of a mechanical stirrer.

This suspension was pumped at 238 kg/h continuously, passing through two heat exchangers in the first part of the tube reactor, with a diameter of 10 to 12 mm, and a second part of the tube reactor with a diameter of 6 to 8 mm.

In the first part of the reactor a flow rate of 29.0 kg/h of oxygen was used.

In the second part of the reactor a flow rate of 11.0 kg/h of oxygen was used and of 12.4 kg/h of rapeseed oil, as a source of fresh carbon dioxide.

The reactor used for the test is sold by the company “Supercritical Fluid Technology Sweden AB” (Karlskoga, Sweden).

After the supercritical and cooling phase, 500 active ppm of formamidinesulfinic acid (CAS 1758-73-2), in a 10% aqueous suspension, relative to the calcium carbonate, was introduced into the product at 90° C.

### Parameters of the Procedure:

| | |
|---|---|
| Supply flow rate of Ca(OH)₂ suspension: | 238 kg/h |
| Temperature/pressure in the supply tank | 20° C./1 bar |
| Temperature/pressure in the heat exchangers | 255-367° C./237 bar |
| Temperature/pressure in the first part of the | 473-569° C./235 bar |
| reactor | |
| Temperature/pressure in the second part of the | 534-567° C./231 bar |
| reactor | |
| Temperature/pressure in the heat exchangers | 364-380° C./229 bar |
| Temperature/pressure at the output of heat | 271° C./229 bar |
| exchangers 1 | |
| Temperature/pressure at the output of heat | 59° C./226 bar |
| exchangers 2 | |

The Applicant indicates that the device in fact has 2 heat exchangers, noted 1 and 2.

The product was then cooled to 16° C. and the pressure reduced to atmospheric pressure.

The product obtained, recovered in a final tank, is a precipitated calcium carbonate according to the invention, of which the rate of nuclear transformation from carbon ¹⁴C into ¹²C is greater than 850 transformations per hour and per gram.

Part of this product was then dried at 120° C. with a view to being analysed. The precipitated calcium carbonate obtained is of pure calcite structure, after infrared and XRD analysis.

Illustration n^{o} 6 is a photograph taken by scanning electron microscope of the product obtained.

A second part of sample in the form of an aqueous suspension was treated with a permanent magnet covered with Teflon.

After 5 minutes' treatment by magnetic separation, the magnet was covered with black and coloured magnetic and/or paramagnetic impurities, which were visible on the surface.

Illustration n^{o} 7 is a photograph taken by a scanning electron microscope of the magnet after the 5 minutes' treatment.

A third part of the sample was ground in an aqueous medium, at a concentration equal to 18% by dry weight of calcium carbonate, without dispersing agent, with balls of zirconium oxide, for 1 hour.

The product obtained was then dispersed in the presence of sodium polyacrylate, and its distribution of granulometric sizes was determined using a device of the Sedigraph™ 5100 type: 62% by weight of the particles had a diameter of less than 2 μm, and 31% by weight of the particles had a diameter of less than 1 μm.

1-21. (canceled)

22. A synthetic mineral matter containing carbonate characterized in that it has a rate of nuclear carbon transformation from ¹⁴C into ¹²C of between 450 and 890 transformations per hour and per gram.

23. The synthetic mineral matter according to claim 22, wherein the rate of nuclear carbon transformation from ¹⁴C into ¹²C is between 700 and 890 transformations per hour and per gram.

24. The synthetic mineral matter according to claim 22, wherein the rate of nuclear carbon transformation from ¹⁴C into ¹²C is between 850 and 890 transformations per hour and per gram.

25. The synthetic mineral matter according to claim 22, wherein the carbonate is chosen from carbonates with monovalent and/or bivalent and/or trivalent cations, or their mixtures.

26. The synthetic mineral matter according to claim 25, wherein said monovalent and/or bivalent and/or trivalent cations are chosen from cations of the first or second or third main group of the Mendeleev periodic table.

27. The synthetic mineral matter according to claim 26, wherein said cations are chosen from lithium, sodium, potassium, magnesium, calcium, strontium or their mixtures.

28. The synthetic mineral matter according to claim 22, wherein the calcium carbonate is a calcium carbonate having a crystalline structure of the calcite or aragonite or vaterite type, or the carbonate is a mixture of a calcium carbonate of structure of the calcareous type with a calcium carbonate of structure of the aragonite type and/or a calcium carbonate of structure of the vaterite type.

29. The synthetic mineral matter according to claim 28, wherein the calcium carbonate is a calcium carbonate that it is a mixture of calcium carbonate of structure of the calcite type and of structure of the aragonite type.

30. The synthetic mineral matter according to claim 22, wherein the carbonate has a degree of whiteness of over 80% TAPPI, determined according to norm TAPPI T452 ISO 2470.

31. The synthetic mineral matter according to claim 22, wherein the carbonate has a degree of whiteness of over 90% TAPPI, determined according to norm TAPPI T452 ISO 2470.

32. The synthetic mineral matter according to claim 22, wherein the carbonate has a degree of whiteness of over 93% TAPPI, determined according to norm TAPPI T452 ISO 2470.

33. The synthetic mineral matter according to claim 22, which is a mixture and/or a costructure with other types of mineral matter chosen from natural and/or synthetic silicas, silicates, clay, talc, mica, aluminium hydroxides, sulphates, satin whites, phosphates, brushites, octacalcium phosphates or hydroxyapatites, or their mixtures.

34. A process for manufacture of synthetic mineral matter containing carbonate, characterized in that it uses carbon dioxide resulting from aerobic or anaerobic fermentation.

35. The process according to claim 34, wherein the carbon dioxide results from anaerobic fermentation.

36. The process according to claim 34, which further comprises using a mixture of carbon dioxide derived from aerobic or anaerobic fermentation, with carbon dioxide from another source.

37. The process according to claim 36, wherein the carbon dioxide from another source is old carbon dioxide.

38. The process of claim 36, wherein the carbon dioxide from another source is from a thermal decomposition of calcium carbonate.

39. The process according to claim 37, wherein the mixture uses less than 50% by weight of old carbon dioxide.

40. The process according to claim 36, wherein the carbon dioxide derived from aerobic or anaerobic fermentation is fresh carbon dioxide derived from the fermentation of sugars or from the combustion of alcohol, ethanol, methanol, alkanes, methane, ethane, or other alkanes originating from the fermentation of organic compounds, fruit, fruit alcohols, or waste from public discharges.

41. The process according to claim 40, wherein the fresh carbon dioxide is a mixture of fresh carbon dioxide derived from the fermentation of sugars with fresh carbon dioxide derived from the combustion of organic compounds or carbon dioxide derived from the fermentation of waste from discharges under supercritical pressure.

42. A process for manufacture of synthetic mineral matter containing carbonate, characterized in that it uses fresh carbon dioxide derived from the thermal decomposition or degradation by oxidation of waste from discharges under supercritical pressure.

43. The process according to claim 42, wherein the carbon dioxide is used at a temperature of between 5° C. and 100° C.

44. The process according to claim 42, wherein the carbon dioxide is used at a temperature of between 20° C. and 30° C.

45. The process according to claim 34, which is a batch process, a continuous process, or a continuous-batch mixture.

46. The process according to claim 45, which is a continuous process with one or more stages of chemical treatment.

47. The process according to claim 46, wherein the chemical treatment is selected from treatment with sodium silicate, treatment with sodium silicate followed by the addition of acid, citric acid, phosphoric acid or other H₃O⁺ donors.

48. The process according to claim 46, wherein the chemical treatments are applied continuously after the carbonic acid treatment.

49. The process according to claim 45, which is a batch process in which the reaction takes place in a single tank into which all the reagents are introduced.

50. The process according to claim 45, which is a continuous process in which fresh carbon dioxide used, or a mixture of fresh carbon dioxide with old carbon dioxide used, is introduced in a cascade of n reactors installed in series and/or in parallel, where n designates a number between 1 and 50.

51. The process according to claim 50, wherein n is between 1 and 10.

52. The process according to claim 50, wherein n is between 1 and 5.

53. The process according to claim 45, which is a continuous-batch mixture process in which a continuous synthesis process is followed by a number m of batch stages, where said stages are the addition of carbon dioxide to the storage or a stage of physical treatment or a stage of chemical treatment or o stages corresponding to the introduction of a dispersing agent, where m designates a number between 1 and 5, and where o designates a number between 0 and 3.

54. The process according to claim 53, wherein the chemical treatment is a treatment with sodium silicate followed by the addition of an acid.

55. The process according to claim 34, wherein said process comprises at least one stage of dispersion and/or at least one stage of grinding.

56. The process according to claim 55, wherein said at least one stage of dispersion and/or at least one stage of grinding is performed in the presence of a least one dispersing agent and/or at least one grinding aid agent.
